(19) United States
(12) Patent Application Publication
(10) Pub. No.: US 2012/0321729 A1
(43) Pub. Date: Dec. 20, 2012

(54) NUTRITIONAL FORMULA FOR MANAGING CHRONIC OBSTRUCTIVE PULMONARY DISEASE (COPD) AND LUNG HEALTH

(76) Inventor: Ven Subbiah, Garner, NC (US)

(21) Appl. No.: 13/492,995

(22) Filed: Jun. 11, 2012

Related U.S. Application Data
(60) Provisional application No. 61/497,101, filed on Jun. 15, 2011.

(51) Int. Cl. A61K 36/537; A61P 11/06; A61P 11/00

(52) U.S. Cl. 424/746

(57) ABSTRACT

The present invention is a novel nutritional product composition formulated to be used as medical foods for managing COPD. The formula comprises clary sage root extract, L-Leucine, L-Isoleucine, L-Valine and L-Glutamine. The most active chemicals that import antioxidants proprieties are Tanshinones. The composition enhances the activity of Phase 2 enzymes that deactivate and react to oxygen species and many toxins present in tobacco smoke. Preparation of highly enriched Tanshinones improved the lung function in patients with chronic asthmatic bronchitis.

This application claims priority of U.S. provisional application No. 61/497,101 filed on Jun. 15, 2011 and is included herein in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention generally relates to dietary supplements, and more particularly to a medical food formula for improving lung health and treating chronic obstructive pulmonary disease (COPD), or chronic asthmatic bronchitis comprising clary sage root extract, L-Leucine, L-Isoleucine, L-Valine, and L-Glutamine, for use as a nutritional supplement, medical food, to manage COPD, and lung health agent.

2. Description of the Related Art

The most important processes causing lung damage during COPD include oxidative stress produced by very high concentrations of oxidants in tobacco smoke. SP001 can restore oxidative balance in smokers, ex-smokers, and other patients with COPD and improve their quality of life. It has been shown that Tanshinone IIA alters regulatory elements involved in oxidative stress and inflammation such as Nrf2 (nuclear factor erythroid 2-related factor 2). Nrf2 enhances the activity of a number of proteins, known as phase 2 enzymes that directly destroy ROS, or deactivate many toxic chemicals present in tobacco smoke. It has been previously disclosed that Tanshinone IIA protects primary rat tracheal epithelial cells from benzopyrene a chemical found in tobacco smoke. Tanshinone IIA also protects lungs from lipopolysaccharide-induced injury in rats and mice.

Cigarette smoke contains more than 10¹⁵ oxidants/free radical molecules per puff and 4,700 chemicals (Church D F, Pryor W A; Enviorn Health Perspect 1985; 64:111-126), this adds to the oxidant burden in smokers. The reactive oxidant species (ROS) generated by smoking induce inflammation in the lung and its airways as well as cause mutations in airway epithelial cell DNA. There is considerable evidence that an increased oxidative burden in the lungs of smokers and ex-smokers can cause direct injury to lung cells, mucus hypersecretion, enhanced lung inflammation, lung cancer, and COPD. The risk of developing these diseases does not disappear after smoking has been discontinued. For example, the local lung inflammation initiated by ROS in cigarette smoke persists in subjects with COPD after they have stopped smoking, explaining the persistence and progression of the disease after smoking has been discontinued (Irfan Rahman Ther Adv Respir Dis. 2008 December; 2(6): 351-374).

ROS form as a natural by-product of the normal metabolism of oxygen and plays important roles within the cell. Under normal circumstances the cell is able to maintain an adequate homeostasis between the formation of ROS and its removal through particular enzymatic pathways or via antioxidants (Irfan Rahman Ther Adv Respir Dis. 2008 December; 2(6): 351-374). However, smoking introduces extremely high concentration of oxidants and overwhelms the antioxidant mechanisms in the cell.

Clary sage is cultivated mainly for the production of essential oil, sclareol and sclareol derivatives used in food products. Traditionally, sage is used as a tonic, digestive, antiseptic, astringent, and antispasmodic. Clary sage has also been used in the treatment of cancer. The most active chemicals that impart antioxidant and anti-inflammatory properties to Clary sage roots (rhizome) are Tanshinones. Tanshinones are orange-red pigments initially reported from the rhizomes of Salvia miltiorrhiza Bunge (Dan-Shen). Dan-Shen has been used for centuries in traditional Chinese medicine for the treatment of coronary heart disease, particularly angina pectoris and myocardial infarction (Adams J D et al. 2006, Preclinical and clinical examinations of Salvia miltiorrhiza and its tanshinones in ischemic conditions, Chin Med Nov, 23:1-3). Tanshinones have common structural features of planar phenanthrene quinine that are comprised of four compounds, Tanshinone I, Tanshinone IIA, Tanshinone IIB, and Cryptotanshinone.

### BRIEF SUMMARY OF THE INVENTION

One embodiment of the present invention is a composition for treating a patient with COPD, or chronic asthmatic bronchitis comprising:
from 1 mg to 3.00 mg of clary sage root extract enriched with Tanshinones, particularly, Tanshinone II A;
from 1 mcg to 1,000 mcg of L-Leucine;
from 1 mcg to 1,000 mcg of L-Isoleucine; and
from 1 mcg to 1,000 mcg of L-Valine.

Another embodiment of the present invention is a medical food, as a beverage or food bar composition for use as a nutritional supplement, Chronic Obstructive Pulmonary Disease (COPD) and lung health agent, comprising:
250 mg of clary sage root extract (SPOO1, Tanshinones);
300 mg of L-Leucine;
150 mg of L-Isoleucine;
150 mg of L-Valine; and
500 mg of L-Glutamine.

Another embodiment of the present invention is a method for treating COPD or CAB comprising administering to a patient in need thereof a composition of the formula comprising:
250 mg of clary sage root extract (SPOO1, Tanshinones);
300 mg of L-Leucine;
150 mg of L-Isoleucine;
150 mg of L-Valine; and
500 mg of L-Glutamine.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### Definitions

The terms “about” and “essentially” mean ±10 percent.

The term “comprising” is not intended to limit inventions to only claiming the present invention with such comprising language. Any invention using the term comprising could be separated into one or more claims using “consisting” or “consisting of” claim language and is so intended.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

Clary sage root extract enriched with Tanshinones (e.g. SP001) is believed to help maintain an oxidative balance in smokers or ex-smokers by providing the additional nutritional requirements of antioxidants or antioxidant mechanisms. SP001 includes phase 2 enzymes, which neutralize reactive electrophiles and act as indirect antioxidants, and are an effective means for achieving protection against ROS generated by smoking. SP001 is a specially formulated medical food product, consisting primarily of a proprietary standardized extract of clary sage, a GRAS plant. Clary sage (Salvia sclarea) has been used extensively in traditional medicines and cultivated in North Carolina.

Though a large number of dietary supplements and medical foods are currently known and marketed, no such supplements include a novel mixture of Clary sage root extract and L-Arginine so as to enhance the Phase II enzyme and restore the lung health. We and others have observed that Tanshinone IIA, the main ingredient in SP001 alters regulatory elements involved in oxidative stress and inflammation such as Nrf2 (nuclear factor erythroid 2-related factor 2). Nrf2 enhances the activity of a number of proteins, known as phase 2 enzymes that directly destroy ROS, or deactivate many toxic chemicals present in tobacco smoke, thereby buttressing the innate defenses of the lung cell to slow accumulation of genetic alterations responsible for the development and progression of the disease. This new strategy provides a marked amplification of the action of chemoprotective agents, since the small molecules that induce Nrf2 activity are not themselves consumed in the deactivation process, thus a single molecule can effectively neutralize the harmful effects of a much larger number of potent mutagenic molecules (Sporn, M B and Liby, K T Nat Clin Pract Oncol, 2005; 2(10): 518-525). Tanshinone IIA is an inducer of heme oxygenase 1 and suppressor of the induction of inducible nitric oxide synthase, which could result in desirable effects on two important gaseous signaling molecules, namely carbon monoxide and nitric oxide that have significant anti-inflammatory properties (Zhang H S, Wang S Q Biochem Pharmacol. 2007 May 1; 73(9):1358-66). The present invention overcomes these and other deficiencies associated with the prior art.

The present invention comprises of a formula for use as a dietary supplement, dietary ingredient, and medical food. The formula is intended for use as a nutritional supplement, COPD, lung health agent. The manner in which the formula provides the specific effects will be discussed in greater detail below. The formula comprises clary sage root extract (enriched for Tashinones), L-Leucine, L-Isoleucine, L-Valine and L-Glutamine.

In one embodiment “the present formula is formulated in food bar, tablet, or capsule form though it will be appreciated that such may also be provided in powder or liquid form. For the desired results, the proper dosages are from one (1) to five (5) food bars or beverage of 2 ounces daily.”

It has been observed that Tanshinone IIA, the main ingredient in SP001 alters regulatory elements involved in oxidative stress and inflammation such as Nrf2 (nuclear factor erythroid 2-related factor 2). Nrf2 enhances the activity of a number of proteins, known as phase 2 enzymes that directly destroy ROS, or deactivate many toxic chemicals present in tobacco smoke, thereby buttressing the innate defenses of the lung cell to slow accumulation of genetic alterations responsible for the development and progression of the disease. This new strategy provides a marked amplification of the action of chemoprotective agents, since the small molecules that induce Nrf2 activity are not themselves consumed in the deactivation process, thus a single molecule can effectively neutralize the harmful effects of a much larger number of potent mutagenic molecules. Tanshinone IIA is an inducer of heme oxygenase 1 and suppressor of the induction of inducible nitric oxide synthase, which could result in desirable effects on two important gaseous signaling molecules, namely carbon monoxide and nitric oxide that have significant anti-inflammatory properties.

Repeated ingestion of glutamine and glutamate showed different effects on the plasma amino acid concentration. Ingestion of glutamine increased the plasma concentrations of citrulline and arginine, substrates produced in the intestine and the liver. Repeated ingestion of 30 mg of glutamine per kilogram of body weight every 20 minutes can increase glutamate availability in healthy elderly and patients, who are likely more dependent on external glutamate ingestion than are young adults.

In accordance with an embodiment of the present invention, there is provided a medical food formula for use as a nutritional supplement, COPD formula and lung health agent. The formula preferably comprises Clary sage root extract (SP001, Tanshinones), 250 mg of Clary sage root extract (SP001), 300 mg L-Leucine, 150 mg L-Isoleucine, 150 mg L-Valine and 500 mg of L-Glutamine.

L-Leucine, L-Isoleucine, L-Valine and L-Glutamine compounds utilized in the aforementioned formula, have been shown to share several key suggest safety and efficacy. The endothelial modulation with these compounds not only has the potential to make angiogenesis effective, but also may have implications for cell therapy trials. The vitamins and niacin is also included in the formula in order to enhance the activity of Phase 2 enzymes that deactivate the reactive oxygen species. Importantly, the additional ingredients such as L-Leucine, L-Isoleucine, L-Valine and L-Glutamine compounds, restore vasicular NO (nitric oxide) activity, facilitate vasodilation, and increase resistance to atherosclerosis and thrombosis. In one embodiment the formula is taken with meals or whenever there is an increase in the blood pressure. Additionally, the formula is, in one embodiment, provided in the form of beverage or a food bar form though it may also be provided in powder or tablet form.

The formulation in one embodiment includes from 1 mg to 3,000 mg of Clary sage root extract enriched with Tanshinones, particularly, Tanshinone II A, from 1 mcg to 1,000 mcg of L-leucine, from 1 mcg to 1,000 mcg of L-Isoleucine, and from 1 mcg to 1,000 mcg of L-Valine.

A specific embodiment includes 250 mg of Clary sage root extract (SP001, Tanshinones), 300 mg of L-Leucine, 150 mg of L-Isoleucine, 150 mg of L-Valine, and 500 mg of L-Glutamine.

The method of the invention includes a method for treating COPD or CAB comprising administering to a patient in need thereof a composition of the formula comprising 250 mg of Clary sage root extract (SP001, Tanshinones), 300 mg of L-Leucine, 150 mg of L-Isoleucine, 150 mg of L-Valine, and 500 mg of L-Glutamine.

### Example of Clary Sage Formulation (Salvia sclarea) Enriched for Tanshinones

Clary sage roots were cut into small pieces and were dried in a drying oven at 45 degrees C. to remove all traces of moisture. The dried material was stored at −20 degrees C. before processing. Dried samples were pulverized using a Wiley Mill with fillers to obtain a 20-40 micron particle size. Samples were stored at −20 degrees C. in air tight ziplock bags.

The sample was systematically extracted using hexane and the hexane fractional washed with 5 percent sodium carbonate. Solvents were then removed with a rotating evaporator. The isolated fraction tests high for Tanshinones (designated SP001). Fingerprinting confirmed analysis using LC-MS. The enriched Clary sage fraction was re-chromotographed on a hypersil C18 reversed phase column (100×2.1 mm, 5 micrometers) eluted with water-acetone gradient with a flow rate of 0.6 ml/min. Sample was monitored based on the UV character as well as mass detection. The LC/MS data was used to quantify Tanshinones IIA concentration in SP001.

Those skilled in the art to which the present invention pertains may make modifications resulting in other embodiments employing principles of the present invention without departing from its spirit or characteristics, particularly upon considering the foregoing teachings. Accordingly, the desired embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

What is claimed is:

1. A composition for treating a patient with COPD, or chronic asthmatic bronchitis comprising:
from 1 mg to 3,000 mg of Clary sage root extract enriched with Tanshinones, particularly, Tanshinone II A;
from 1 mcg to 1,000 mcg of L-Leucine;
from 1 mcg to 1,000 mcg of L-Isoleucine; and
from 1 mcg to 1,000 mcg of L-Valine.

2. The composition of claim 1 comprising 1 to 1000 mg of L-Glutamine.

3. A medical food, as a beverage or food bar composition for use as a nutritional supplement, Chronic Obstructive Pulmonary Disease (COPD) and lung health agent, comprising:
250 mg of clary sage root extract (SP001, Tanshinones);
300 mg of L-Leucine;
150 mg of L-Isoleucine;
150 mg of L-Valine; and
500 mg of L-Glutamine.

4. The composition of claim 3 wherein said formula is in the form of a beverage.

5. The formula of claim 3 wherein said composition is in the form of a powder.

6. The formula of claim 3 wherein said composition is in the form of a liquid.

7. The formula of claim 3 wherein said composition is in the form of a food bar.

8. The formula of claim 3 wherein said composition is in the form of a tablet.

9. A method for treating COPD or CAB comprising administering to a patient in need thereof a composition of the formula comprising:
250 mg of clary sage root extract (SP001, Tanshinones);
300 mg of L-Leucine;
150 mg of L-Isoleucine;
150 mg of L-Valine; and
500 mg of L-Glutamine.
